(19) United States
(12) Patent Application Publication
(10) Pub. No.: US 2022/0319660 A1
(43) Pub. Date: Oct. 6, 2022

(54) DETECTION OF THE SYNCHRONIZATION BETWEEN THE ACTUATION OF A METERED-DOSE INHALER AND A PATIENT'S INSPIRATION

(71) Applicants: HEPHAÏ, Paris (FR); ASSISTANCE PUBLIQUE HOPITAUX DE PARIS (AP-HP), Paris (FR); SORBONNE UNIVERSITE, Paris 6 (FR); INSTITUT NATIONAL DE LA SANTE ET DE LA RECHERCHE MEDICALE (INSERM), Paris (FR)

(72) Inventors: Isabelle Geneviève BELANGER-BASSET, Malakoff (FR); Jean-Pierre Louis Ludovic MINVIELLE, Bordeaux (FR); Valéry TROSINI DESERT, Paris (FR); Thomas SIMILOWSKI, Issy Les Moulineaux (FR)

(21) Appl. No.: 17/709,874

(22) Filed: Mar. 31, 2022

(30) Foreign Application Priority Data
Apr. 1, 2021 (FR) 2103413

(51) Int. Cl. G16H 20/13; A61M 15/00; G06V 20/40; G06V 40/20; G06V 10/80; G06T 7/20; G06T 7/70; G10L 21/14; G06N 3/08

(52) U.S. Cl. CPC G16H 20/13; A61M 15/0068; A61M 15/0021; G06V 20/46; G06V 40/28; G06V 10/80; G06T 7/20; G06T 7/70; G10L 21/14; G06N 3/08; G06T 2207/10016

(57) ABSTRACT

A pressurized metered-dose inhaler requires good synchronization between the activation of the inhaler and the inspiration by the patient. Processing is carried out on the video frames filming the patient to qualify the actuation of the pressurized metered-dose inhaler according to two criteria: regarding the pressing by the patient's actuating fingers on a trigger member of the inhaler and regarding the actual compression of the inhaler. Processing of an audio signal recording the patient at the same time is also carried out to detect an inhalation by the patient. A temporal correlation of the probabilities obtained as results then makes it possible to qualify the synchronization between the actuation of the inhaler by the patient and the latter's inspiration, and thereby indicate a proper use or improper use of the inhaler.

### CROSS-REFERENCE TO RELATED APPLICATIONS

This application claims priority to FR 2103413 filed Apr. 1, 2021, the entire contents of which are hereby incorporated by reference.

### BACKGROUND OF THE INVENTION

### Field of the Invention

The invention concerns the tracking of the use or utilization of a metered-dose inhaler by a patient subjected to an inhaled therapeutic treatment, typically medication-based.

### Description of the Related Art

The cornerstone of the treatment of asthma and chronic obstructive pulmonary disease, COPD, is based on ready-to-use inhalers prescribed for long-duration use.

Proper use of inhalation devices is crucial to the relief of the symptoms of asthma and of COPD and to the prevention of exacerbations of these diseases. Proper adherence to taking by the inhaler and proper use of the inhaler are two fundamental components for a good level for treatment effectiveness.

30 to 40% of patients do not know how to use their inhaler properly. This is referred to as misuse. The latter has non-negligible medical and economic consequences. It is thus countered.

Document US 2013/063579 describes a system for detecting the proper actuation of an inhaler combining video and audio processing. The video is processed to check the positioning of the face of the user-patient, the proper positioning of the inhalation device, then the actuation of the inhaler. This actuation is confirmed using analysis of a recorded audio signal, in which a target sound is sought. An audio recognition system may also be used, which is trained to classify different sounds, for example inhalation sounds with or without teeth disturbing the stream of air, which may possibly be according to the volume of air drawn in.

From document WO 2019/122315 there is also known a system and a method which use a neural network applied to video and audio signals, to detect the type of aerosol inhaler and any disparity in its use, including the patient's posture, the positioning of the inhaler or for instance the patient's breathing such as improper synchronization of the actuation of the inhaler.

The synchronization between the actuation of the aerosol inhaler and the patient's inspiration is crucial for proper taking of medication. It is in particular challenging to perform and thus to check for pressurized metered-dose inhalers. The known automatic techniques do not make it possible to detect the misuse resulting from desynchronization as accurately as the medical professional observing the patient.

There is thus a need to improve these techniques to enable better detection of the misuse of pressurized metered-dose inhalers that is autonomous and thereby better educate patients in proper taking of medication, while limiting the intervention of medical professionals.

### SUMMARY OF THE INVENTION

The invention thus provides a computer-implemented method for tracking use, by a patient, of a pressurized metered-dose inhaler, comprising the following steps:

obtaining a video signal and an audio signal of a patient using a pressurized metered-dose inhaler,

calculating, for each of a plurality of video frames of the video signal, at least one from among a so-called pressing probability, that an actuating finger of the patient in the video frame is in a phase of pressing on a trigger member of the pressurized metered-dose inhaler, and a so-called compression probability, that the pressurized metered-dose inhaler in the video frame is in a compressed state,

calculating, for each of a plurality of audio segments of the audio signal, a so-called inhalation probability, of the patient performing, in the audio segment, an inspiration combined with the aerosol stream,

determining a degree of synchronization between the actuation of the pressurized metered-dose inhaler and an inspiration by the patient from the pressing, compression and inhalation probabilities corresponding to same instants in time, and

accordingly issuing to the patient a signal of proper use or misuse of the pressurized metered-dose inhaler.

The inventors have noted the effectiveness, in terms of detecting the synchronization, of combined taking into account of a video probability (for detection) of mechanical action on the pressurized metered-dose inhaler (via the actuating fingers and/or via the actual compression of the inhaler) and an audio probability (of detection) of an inhalation or inspiration by the patient.

Computerized calculation techniques make it possible to obtain such probabilities efficiently, by processing video and audio signals.

In a complementary manner, the invention also relates to a computer system comprising one or more processors, for example a CPU processor or processors and/or a graphics processor or processors GPU and/or a microprocessor or microprocessors, which are configured for:

obtaining a video signal and an audio signal of a patient using a pressurized metered-dose inhaler,

calculating, for each of a plurality of video frames of the video signal, at least one from among a so-called pressing probability, that an actuating finger of the patient in the video frame is in a phase of pressing on a trigger member of the pressurized metered-dose inhaler, and a so-called compression probability, that the pressurized metered-dose inhaler in the video frame is in a compressed state,

calculating, for each of a plurality of audio segments of the audio signal, a so-called inhalation probability, of the patient performing, in the audio segment, an inspiration combined with the aerosol stream,

determining a degree of synchronization between the actuation of the pressurized metered-dose inhaler and an inspiration by the patient from the pressing, compression and inhalation probabilities corresponding to same instants in time, and

accordingly issuing to the patient a signal of proper use or misuse of the pressurized metered-dose inhaler.

This computer system may simply take the form of a user terminal such as a smartphone, a digital tablet, a portable computer, a personal assistant, an entertainment device (e.g. a games console), or for instance a fixed device such as a desktop computer or more generally an interactive terminal, for example disposed at home or in a public space such as a pharmacy or a medical center.

Optional features of the invention are defined in the dependent claims. Although these features are mainly set out below in terms of method, they may be transposed into system or device features.

According to one embodiment, determining a degree of synchronization comprises determining, for each type of probability, a temporal window of high probability, and the degree of synchronization is a function of a temporal overlap between the temporal windows so determined for the probabilities.

A temporal correlation of the determined probabilities is thus obtained at low cost.

According to another embodiment, determining a degree of synchronization comprises:

combining (e.g. linearly), for each of a plurality of instants in time, the probabilities of pressing, of compression and of inhalation corresponding to said instant in time into a combined probability, and

determining, from the combined probabilities, a degree of synchronization between the actuation of the pressurized metered-dose inhaler and an inspiration by the patient.

Thus, the steps of detecting (through the three probabilities) are correlated and unified into a single detection function which can easily be optimized.

In one embodiment, the method further comprises a step consisting of comparing the combined probabilities with a threshold value of proper synchronization.

In one embodiment, calculating a pressing probability for a video frame comprises:

detecting, in the video frame, points representing the actuating finger, and

determining a relative descending movement of the tip of the actuating finger relative to a base of the finger, compared to at least one temporally preceding video frame,

the pressing probability being a function of the amplitude of the descending movement from a starting position determined in a preceding video frame.

The direct taking into account of the user's action gives improved detection.

According to a feature, calculating a pressing probability for a video frame comprises a step consisting of comparing the amplitude of the movement to a dimension of the pressurized metered-dose inhaler in the video frame. The real dimension (length) of the inhaler is put to the scale of its dimension in the video frame in particular in order to know the maximum amplitude of movement possible in the video frame and thereby determine the degree (and thus a probability) of the pressing made by the patient.

In one embodiment, calculating a compression probability for a video frame comprises:

comparing a length of the pressurized metered-dose inhaler in the video frame with a reference length of the pressurized metered-dose inhaler, generally in a preceding video frame.

Again, in addition to the true length (dimension) of the inhaler, its theoretical compression stroke may also be put to the scale of their length and stroke in the video frame to enable a comparison to be made for example between the length of the inhaler, its decompressed length (as reference in a preceding frame) and its maximum stroke. A linear approach makes it possible in particular to obtain a probability (between no compression and a maximum compression corresponding to the maximum stroke).

In one embodiment, an audio segment corresponds to a section from 1 to 5 seconds (s) of the audio signal, preferably a section from 2 to 3 s. The audio segments are typically generated with a step size less than their duration. Thus audio segments are generated overlapping in higher or lower number (according to said step size).

In one embodiment, calculating an inhalation probability for an audio segment comprises:

converting the audio segment into a spectrogram, and

using the spectrogram as input to a trained neural network which outputs the inhalation probability. The inventors have noted the effectiveness of modeling spectrograms of the audio signal in the recognition of a patient's inspiration combined with the noise of the aerosol stream.

In a variant, calculating an inhalation probability for an audio segment comprises;

computing a distance between a profile of the audio segment and a reference profile. This distance may then be converted into probability. An audio segment profile may typically be formed from the audio signal itself, from a frequency transform thereof (e.g. a Fourier transform, whether fast or not), from a vector of parameters, in particular MFCC parameters, MFCC standing for Mel-Frequency Cepstral Coefficients.

In one embodiment, the steps consisting of calculating the pressing, compression and inhalation probabilities on later audio segments and video frames are triggered by the detection of proper positioning of the pressurized metered-dose inhaler relative to the patient in earlier video frames. Thus, determining the proper or improper synchronization may be carried out automatically solely for later instants in time, in particular on later video frames.

In another embodiment, the method further comprises an initial determination step for determining opening of the metered-dose inhaler by detecting a characteristic click sound in at least one audio segment of the audio signal, the detection employing a learnt detection model. This determination may possibly be combined with a detection via the video signal. The detection of the opening may in particular constitute an event triggering the subsequent detections, and in particular that of the degree of synchronization by combination of the different calculated probabilities.

The invention also relates to a computer-readable non-transient carrier storing a program which, when it is executed by a microprocessor or a computer system, leads the system to carry out any method as defined above.

Given that the present invention may be implemented in software, the present invention may be incorporated in the form of computer-readable code configured to be supplied to a programmable apparatus on any appropriate carrier. A tangible carrier may comprise a storage medium such as a hard disk, magnetic tape or a semiconductor-based memory device having and others. A transient medium may comprise a signal such as an electrical signal, an electronic signal, an optical signal, an acoustic signal, a magnetic signal or an electromagnetic signal, for example a microwave or RF signal.

### BRIEF DESCRIPTION OF THE DRAWINGS

Still other particularities and advantages of the invention will appear in the following description, illustrated by the appended drawings which illustrate example embodiments that are in no way limiting in character. In the drawings:

FIG. 1 illustrates a system for tracking the use by a patient of a pressurized metered-dose inhaler, according to embodiments of the invention;

FIG. 2 diagrammatically illustrates functional blocks or units of a user device for an implementation of the invention;

FIG. 3 illustrates the interaction between the fingers of a patient and a pressurized metered-dose inhaler;

FIG. 4 illustrates the determination of proper or improper synchronization based on three determined probabilities according to embodiments of the invention;

FIG. 4a illustrates the determination of proper or improper synchronization based on three determined probabilities according to other embodiments of the invention; and

FIG. 5 illustrates, using a flowchart, general steps for the implementation of the invention according to certain embodiments.

### DESCRIPTION OF THE PREFERRED EMBODIMENTS

The proper use of inhaled therapies is essential in the treatment of asthma and of COPD in adults and children. This is generally ensured by compliance with instructions issued by the medical profession, for instance by a doctor.

Aids for taking medication have been developed to educate patients and make them more autonomous in relation to doctors.

Document WO 2019/122315 discloses for example a computer-implemented system for teaching medication taking for a patient using a device for inhaling a therapeutic aerosol, then comment or provide feedback on that medication taking.

As indicated in that document, an inhaler is an inhalation device capable of issuing a therapeutic aerosol enabling a user or a patient to inhale the aerosol. An aerosol is a dispersion of a solid, semi-solid or liquid phase in a continuous gaseous phase, comprising thus for example powder aerosol—known under the pharmaceutical name of powders for inhalation—and mist aerosols. The inhalation devices for the administration of aerosols in powder form are commonly described as powder inhalers. Liquids in aerosol form are administered by means of various inhalation devices, in particular nebulizers, pressurized metered-dose inhalers and soft mist inhalers.

There is a difficulty for the tracking of medication taking in case of the use of pressurized metered-dose inhalers, also designated as pMDI inhalers or pressurized metered-dose aerosols. As a matter of fact, these require particular attention from the patient to the proper synchronization between the actuation of the inhaler and his or her own inspiration, which can be difficult for a patient beginning the treatment or for certain groups of the population.

The pressurized metered-dose inhaler comprises a canister of aerosol liquid inserted into a head (or cartridge mounting) bearing a mouthpiece. The compressing of the inhaler simply by pressing the canister relative to the head, thereby compressing the inhaler, delivers a dose of aerosol which the patient inhales, on exiting the mouthpiece, by inspiration.

The present invention improves the techniques for detecting proper or improper synchronization by analyzing, possibly in real-time or practically in real-time, video and audio signals captured during the medication taking.

Processing is carried out on the video frames filming the patient to qualify the actuation of the pressurized metered-dose inhaler according to two criteria but also based on an audio signal that records the patient at the same time, in order to detect or not detect an inhalation by the patient. A temporal correlation of the results then makes it possible to qualify the synchronization between the actuation of the pressurized metered-dose inhaler and the patient's inspiration, and thereby indicate back to the patient a proper use or improper use of the inhaler.

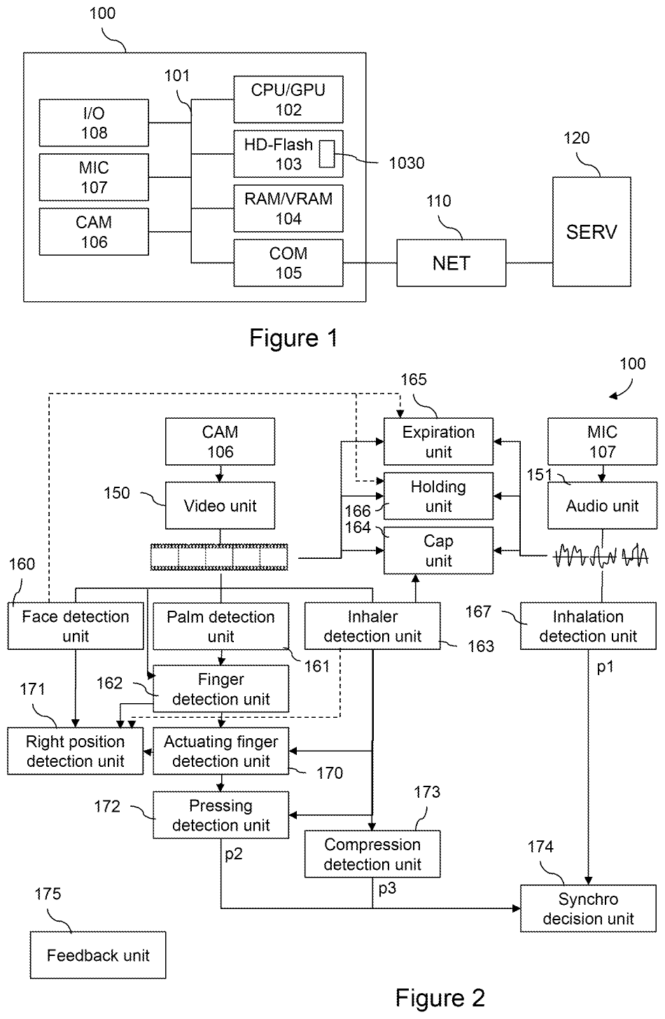

FIG. 1 illustrates a system for tracking the use by a patient of a pressurized metered-dose inhaler, and thus its proper or improper use.

The system comprises a user device 100 configured to implement certain embodiments of the invention. The user device 100 may be a portable device such as a smartphone, a digital tablet, a portable computer, a personal assistant, an entertainment device (e.g. a games console), or may be a fixed device such as a desktop computer an interactive terminal, for example disposed at home or in a public space such as a pharmacy or a medical center. More generally, any computer device suitable for the implementation of the processing operations referred to above may be used.

The device 100 comprises a communication bus 101 to which there are preferably connected:
one or more central processing units 102, such as processors CPU and/or graphics processors or cards GPU and/or one or more microprocessors;
a storage memory 103, of ROM and/or hard disk and/or flash memory type, for the storage of computer programs 1030 configured to implement the invention and in addition for the storage of any data required to run the programs;
a volatile memory 104, of RAM or even video RAM (VRAM) type, for the storage of the executable code of the computer programs as well as registers configured to record variables and parameters required for their execution;
a communication interface 105 connected to an external network 110 in order to communicate with one or more remote servers 120 in certain embodiments of the invention;
a video capture device 106, typically an integral or mounted-on camera, able to capture a video sequence or signal of the patient using the pressurized metered-dose inhaler. The video capture device 106 may be formed by a single camera or by an array of cameras. Typically, a video capture device 106 has an image frequency (or frame rate) of 20, 25, 30, 50 or more frames per second;
an audio capture device 107, typically an integral or mounted-on microphone, able to capture an audio sequence or signal of the patient using the pressurized metered-dose inhaler. The audio capture device 107 may be formed by a single microphone or by an array of microphones.
one or more complementary inputs/outputs I/O 108 enabling the patient to interact with the programs 1030 of the invention in course of running. Typically, the inputs/outputs may include a screen serving as a graphical interface with the patient and/or a keyboard or any other pointing means enabling the patient for example to launch execution of the programs 1030 and/or a loud-speaker. The screen or loud-speaker may serve as an output to provide the patient with feedback on the medication taking as analyzed by the programs 1030 according to the invention.

Preferably, the communication bus provides the communication and the interoperability between the different components included in the computer device 100 or connected thereto. The representation of the bus is non-limiting and, in particular, the central processing unit may be used to communicate instructions to any component of the computer device 100 directly or by means of another component of the computer device 100.

The executable code stored in memory 103 may be received by means of the communication network 110, via the interface 105, in order to be stored therein before execution. As a variant, the executable code 1030 is not stored in non-volatile memory 103 but may be loaded into volatile memory 104 from a remote server via the communication network 110 for execution directly. This is the case in particular for web applications (web apps).

The central processing unit 102 is preferably configured to control and direct the execution of the instructions or parts of software code of the program or programs 1030 according to the invention. On powering up, the program or programs that are stored in non-volatile memory 103 or on the remote server are transferred/loaded into the volatile memory 104, which then contains the executable code of the program or programs, as well as registers for the storage of the variables and parameters required for the implementation of the invention.

In one embodiment, the processing operations according to the invention are carried out locally by the user device 100, preferably in real-time or practically in real-time. In this case, the programs 1030 in memory implement all the processing operations described below.

In a variant, some of the processing operations are performed remotely in one or more servers 120, possibly in cloud computing, typically the processing operations on the video and audio signals. In this case, all or some of these signals, which may be filtered, are sent via the communication interface 105 and the network 110 to the server, which in response sends back certain information such as the probabilities discussed below or simply the information representing the degree of synchronization or for instance the signal to provide back to the patient. The programs 1030 then implement part of the invention, complementary programs provided on the server or servers implementing the other part of the invention.

The communication network 110 may be any wired or wireless computer network or a mobile telephone network enabling connection to a computer network such as the Internet.

FIG. 2 diagrammatically illustrates functional blocks or units of the device 100 for an implementation of the invention. As indicated above, some of these functional units may be provided in the server 120 when some of the processing operations are performed remotely there.

The video unit 150 adjoining the camera or cameras 106 records the video signal captured in one of the memories of the device, typically in RAM memory 104 for processing in real-time or practically in real-time. This recording consists in particular of recording each video frame of the signal. When this occurs, each frame is time-stamped using an internal clock (not shown) of the device 100. The time-stamping enables final temporal correlation of the information obtained by the processing operations described below.

In one embodiment directed to reducing the processing load, a subset only of the frames may be recorded and processed, typically 1 or N−1 frames every N frames (N being an integer, for example 2, 3, 4, 5 or 10).

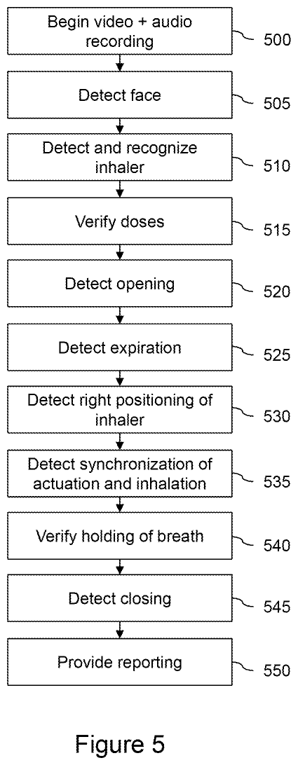

In corresponding manner, the audio unit 151 adjoining the microphone or microphones 107 records the audio signals captured in one of the memories of the device, typically in RAM memory 104. The audio unit 151 can typically pre-process the audio signal for the purposes of creating audio segments for later processing operations. The length (in time) of the segments may vary dynamically according to the processing to apply, thus according to the state of advancement of the algorithm described below (FIG. 5).

For example, segments of 1 second length may be created for processing by unit 164 for detecting the opening or closing of the cap of the pressurized metered-dose inhaler. However, longer segments, typically of 2 to 10 s length, preferably 3 to 5 s, ideally approximately 3 s, are created and stored in memory for processing by the units for detecting expiration 165, inhalation 167 and the holding of breath 166.

Generally speaking, audio segments of length substantially equal to 3 s may be provided for the entire algorithm.

Successive audio segments may overlap. They are for example generated with a generation step between 1/10s and 1 s, for example 0.5 s. Preferably, the audio segments are aligned with video frames, for example the middle of an audio segment corresponds to a video frame (within a predefined tolerance, for example 1/100 s for a frame rate of 25 FPS).

In a manner similar to the video frames, each audio segment is time-stamped, typically with the same label as the corresponding video frame (or the closest one) at the center of the audio segment. Of course, other correspondence between video frame, audio segment and time stamping may be envisioned.

Each video frame is supplied as input to the face detection unit 160, to the palm detection unit 161, to the finger detection unit 162, to the inhaler detection unit 163 and to the unit for detecting the opening or closing of the inhaler 164, optionally to the expiration detection unit 165 and to the breath-holding detection unit 166.

Each audio segment is supplied as input to the unit for detecting the opening or closing of the inhaler 164, to the expiration detection unit 165, to the breath-holding detection unit 166 and to the inhalation detection unit 167.

The face detection unit 160 may be based on known techniques for face recognition in images, typically image processing techniques. According to one embodiment, unit 160 implements an automatic learning pipeline or automatic learning models or supervised machine learning. Such a pipeline is trained to identify 3D facial marker points.

In known manner, a pipeline or supervised automatic learning model may be regression or classification based. Examples of such pipelines or models include decision tree forests or random forests, neural networks, for example convolutional, and support vector machines (SVMs).

Typically, convolutional neural networks may be used for this unit 160 (and the other units below that are based on an automatic learning model or pipeline).

The publication “Real-time Facial Surface Geometry from Monocular Video on Mobile GPUs” (Yury Kartynnik et al) typically describes an end-to-end model based on a neural network to derive an approximate 3D representation of a human face, from 468 marker points in 3D, based on a single camera input (i.e. a single frame). It is in particular well-adapted for processing by graphics cards of mobile terminals (i.e. with limited resources). The 468 marker points in 3D comprise in particular points representing the mouth of the face.

The face detection unit 160 may also be configured to perform tracking (or following) of the face in successive frames. Such tracking makes it possible to resolve certain difficulties of detection in a following image (face partially concealed). For example, the sudden non-detection of a face in a video frame may be replaced by an interpolation (e.g. linear) of the face between an earlier frame and a later frame.

The palm detection unit 161 may also be based on known techniques for hand or palm recognition in images, typically image processing techniques. According to one embodiment, unit 161 implements automatic pipeline learning, for example convolutional neural network based. Such a pipeline is trained to identify 3D hand marker points.

The publication “MediaPipe Hands: On-device Real-time Hand Tracking” (Fan Zhang et al.) describes an applicable solution. Again, the palm detection unit 161 may be configured to perform tracking (or following) in order to correct certain detection difficulties in a given frame.

The finger detection unit 162 is based on detection of the palm by unit 161 to identify and model, for example in 3D, the 3D marker points of the fingers of the hand. Conventional image processing operations may be implemented (searching for hand models in the image around the located palm). According to one embodiment, unit 162 implements automatic pipeline learning, for example convolutional neural network based. Such a pipeline is trained to identify 3D marker points of the fingers.

As input, unit 162 may receive the video frame cropped in the neighborhood of the palm identified by unit 161. This neighborhood or region of interest is known by the term “bounding box”, and is dimensioned to encompass the entirety of the hand for which the palm has been identified.

The above publication “MediaPipe Hands: On-device Real-time Hand Tracking” describes an applicable solution. Again, the finger detection unit 162 may be configured to perform tracking (or following) in order to correct certain detection difficulties in a given frame (for example a hidden finger).

Typically, the 3D marker points of the fingers of the hand comprise the interphalangeal joints (joint at the base of each finger, joints between phalanges) and the finger tips, as well as a link between each of these points, thereby identifying the chain of points forming each finger and enabling the tracking thereof.

The units for palm detection 161 and finger detection 162, although they may be represented as being in the drawing, may be implemented together, for example using a single convolutional neural network based automatic learning pipeline.

The inhaler detection unit 163 may be based on known techniques for recognition of known objects in images, typically image processing techniques. According to one embodiment, unit 163 implements automatic pipeline learning, for example convolutional neural network based. Such a pipeline is trained to identify different inhaler models. It may be created from a partially pre-trained pipeline (for the recognition of objects) and ultimately trained using a set of data specific to inhalers.

Preferably, unit 163 locates the inhaler in the processed video frame (a region of interest or “bounding box” around the inhaler may be defined), identifies a family or model of inhaler (according to whether the learning data have been labeled by specific type or family of inhaler) and optionally its orientation relative to a guiding axis (for example a longitudinal axis for a pressurized metered-dose inhaler).

A regression model produces a score, indicator or probability of confidence/plausibility on a continuous scale (model output). As a variant, a classification model produces a score, indicator or probability of confidence/plausibility on a discrete scale (output from the model corresponding to a type or family of inhaler).

Several models may be used for detecting objects, for example faster R-CNN, Mask R-CNN, CenterNet, EfficientDet, MobileNet-SSD, etc.

The publication “SSD: Single Shot MultiBox Detector” (Wei Liu et al.) for example describes a convolutional neural network model which enables both the location and the recognition of objects in images. Location is in particular possible by virtue of the evaluation of several bounding boxes of sizes and ratios that are fixed at different scales of the image. These scales are obtained by passage of the input image through successive convolutional layers. The model thus predicts both the offset of the bounding boxes with the object searched for and the degree of confidence in the presence of an object.

The inhaler detection unit 163 may be configured to perform the tracking (or following) of the inhaler in successive frames, in order to correct certain difficulties of detection in a given frame.

The unit for detecting the opening or closing of the inhaler 164 makes it possible, when the inhaler is provided with a cap or shutter, to detect whether the latter is in place (inhaler closed) or withdrawn/open.

This unit 164 may operate only on the video frames, or only on the audio segments or on both.

Image processing techniques, based on inhaler models with or without cap/shutter, may be used on the video frames, optionally on the region of interest surrounding the inhaler as identified by unit 163. According to one embodiment, unit 164 implements a convolutional neural network trained to perform classification between an open inhaler and a closed inhaler, in the video frames.

Thus, a switch to an open state (and respectively closed state) is detected when a classification passes from “closed inhaler” for earlier frames to “open inhaler” for later frames. The first later frame may indicate an instant in time of the opening.

Signal processing techniques make it possible, in the audio segments, to identify a sound characteristic of the opening or of the closing of the inhaler, typically a “click” specific to one type of inhaler or one family for inhalers. Audio signal models may be predefined and searched for in the audio segments. As a variant, markers (typically parameters such as Mel-Frequency Cepstral Coefficients) that are typical of these characteristic sounds are searched for in the segments analyzed. According to one embodiment, unit 164 implements a convolutional neural network trained to perform classification between an opening sound and a closing sound of the inhaler, in the audio segments.

The convolutional neural network model is for example trained with spectrograms. Such a classical learning model is for example trained on markers/indicators characteristic of the sound (MFCC for example).

A temporal correlation between the audio segments detecting the opening (and respectively the closing) of the inhaler and the video frames revealing a switch towards an open state (and respectively a closed state) of the inhaler (that is to say a defined number of frames around or just after that switch) makes it possible to confirm or strengthen the level of confidence in the video detection of the opening or closing of the inhaler.

The units for detection of an expiration 165, of a holding of breath 166 and of an inspiration/inhalation 167 analyze the audio segments to detect therein an expiration/a holding of breath/an inspiration or inhalation by the patient.

They may implement simple reference sound models or markers (typically markers/parameters such as Mel-Frequency Cepstral Coefficients) typical of those reference sounds which are searched for in the segments analyzed. According to one embodiment, all or some of these units implement an automatic learning model, typically a convolutional neural network, trained to detect the reference sound. As the three reference sounds, expiration, breath holding and inspiration/inhalation, are different in nature, the three units may be trained in dissociated manner, with distinct data sets.

Preferably, each audio segment is filtered using a high-pass Butterworth filter, of which the cut-off frequency is chosen sufficiently low (for example 400 Hz) to remove hindering components of the spectrum. The filtered audio segment is then converted into a spectrogram, for example into a mel-spectrogram. The learning of the models (e.g. convolutional neuronal networks) is then carried out on such annotated spectrograms (learning data).

A regression model produces a score, indicator or probability of confidence/plausibility on a continuous scale (model output). As a variant, a classification model produces a score, indicator or probability of confidence/plausibility on a discrete scale (model output) which classifies the audio segments into segments that comprise or do not comprise the sound searched for. The result of this is thus what is referred to as a level, score, or indicator of confidence or a probability, of expiration, breath holding or inhalation, that the patient makes, in the audio segment, a prolonged expiration, a holding of breath or an inspiration that is combined with the aerosol stream.

The probability of inhalation is denoted p1 in the Figure.

In a simple version, the automatic learning model for detecting a holding of the breath is the same as that for detecting an expiration, the outputs being interchanged: an absence of expiration is equivalent to the holding of breath, whereas an expiration is equivalent to the absence of the holding of breath. This simplifies the algorithm complexity of units 165 and 166.

In a still simpler version, one and the same non-binary model may be trained to learn several classes: expiration (for unit 165), inspiration (for unit 167), the absence of expiration/inspiration (for unit 166), or even the opening (uncapping) and the closing (capping) of the inhaler (for unit 164). Thus, a probability of each event is accessible via a single model for each processed audio segment.

The unit for detection of an expiration 165 may furthermore comprise video processing suitable for detecting an open mouth.

It may be image processing. For example, unit 165 receives as input the 3D marker points from the face detection unit 160 for the current video frame, and detects the opening of the mouth when the 3D points representing the upper and lower edges of the mouth are sufficiently far apart.

As a variant, an automatic learning model, typically a trained convolutional neural network, is implemented.

A temporal correlation between successive video frames revealing a mouth open for a minimum duration (in particular between 1 and 5 s, for example approximately 3 s) and the audio segments detecting an expiration reference sound makes it possible to confirm or strengthen the confidence level/score/indicator of the audio detection of the expiration.

Similarly, the unit for detecting a holding of breath 166 may furthermore comprise video processing able to detect a closed mouth.

It may be image processing. For example, unit 166 receives as input the 3D marker points from the face detection unit 160 for the current video frame, and detects a closed mouth when the 3D points representing the upper and lower edges of the mouth are sufficiently close.

As a variant, an automatic learning model, typically a trained convolutional neural network, is implemented.

A temporal correlation between successive video frames revealing a mouth closed for a minimum duration (in particular between 2 and 6 s, for example 4 or 5 s) and the audio segments detecting a breath holding reference sound makes it possible to confirm or strengthen the confidence level/score/indicator of the audio detection of the breath holding.

The user device 100 further comprises the actuating finger detection unit 170, the unit for detecting a proper position of the inhaler 171, the unit for detecting pressing 172, the unit for detecting compression 173, the synchronization decision unit 174 and the feedback unit 175.

The unit for detection the actuating finger 170 receives as input the 3D marker points of the fingers (from unit 162) and the information on location of the inhaler in the image (from unit 163).

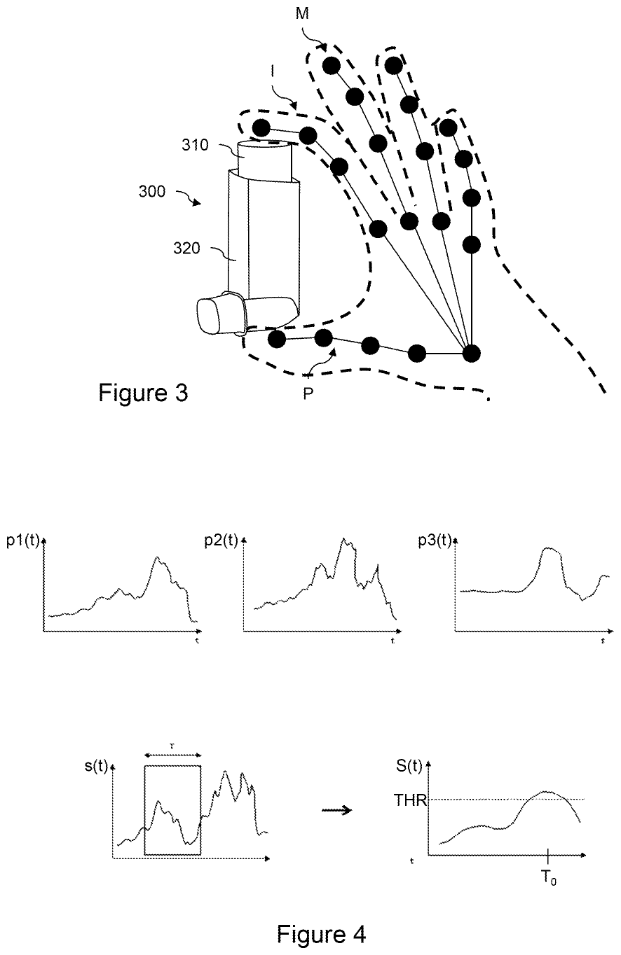

The concern here is with the pressurized metered-dose inhalers that are used in inverted vertical position (opening towards the bottom) as shown in FIG. 3.

The detection of the actuating finger or fingers, that is to say those positioned to actuate the inhaler (in practice to press on the canister 310 relative to the head 320), by unit 170 may be carried out as follows.

The 3D marker points of fingers present in the region of interest around the inhaler (obtained from unit 163) are taken into account and enable a classification of the holding of the inhaler in inverted vertical position (that is to say how the inhaler is held by the patient).

This classification may be made by a simple algorithm revealing geometric considerations or using an automatic learning model, typically a convolutional neural network.

In an algorithm example, unit 170 determines that the thumb tip is located or not located under the head 320 and, in the affirmative, that the end of the index finger is placed on the bottom of the canister 310. This is the case when the 3D marker point of the thumb end is detected as substantially located in the neighborhood of and below the inverted head 320 while the end of the index finger is detected as substantially located in the neighborhood of and above the inverted canister 310. This holding corresponds to a first class C1.

Other classes Ci, which are predefined and in a specific number, may be detected, for example by way for illustration that is not exhaustive:

C2: thumb tip under the head 320 and the end of the index finger on the canister bottom 310,

C3: thumb tip under the head 320 and the ends of the index and middle finger on the canister bottom 310,

C4: index finger end on the canister bottom 310, the other fingers surrounding the head,

C5: middle finger end on the canister bottom 310, the other fingers surrounding the head,

C6: inhaler held with both hands, ends of the right-hand index and middle finger on the bottom of the canister 310, etc.

With each class there is associated an actuating finger, typically the finger or fingers placed on the bottom of the canister 310. This information is stored in memory. Unit 170 performing the classification of the manner of holding the inhaler is thus capable for yielding, as output, the actuating finger or fingers

For example, for class C1, the actuating finger is the index finger “I”. For class C2, this is the middle finger “M’. For class C3, there are two actuating fingers; the index and middle fingers.

The unit for detecting proper position of the inhaler 171 performs processing of the information obtained by units 160 (position of the face and of the mouth), 162 (position of the fingers), 163 (position and orientation of the inhaler) and 170 (actuating finger).

The detection of the proper or improper positioning of the pressurized metered-dose inhaler may simply consist of classifying (proper or improper positioning) a video frame by also taking into account the class Ci of inhaler holding.

This classification may be made by a simple algorithm revealing geometric considerations or using an automatic learning model, typically a convolutional neural network.

In an algorithm example, for classes C1-C3, it is checked whether the hand is placed vertically with the thumb downward, that is to say the 3D marker point of the thumb tip “P” is located further down than that of the actuating fingers (index finger “I” and/or middle finger “M”), and the distance between the 3D marker point of the tip for the actuating finger or fingers and the 3D marker point of the thumb tip “P” is greater than a threshold value (function of the dimension of the inhaler determined for example by unit 163 identifying the inhaler type or family in the video frames).

Furthermore, the 3D marker point of the thumb tip “P” must not be located further down than a certain threshold measured from the 3D marker point of the middle points of the mouth as supplied by unit 160 and/or the bottom of the head 320 of the inhaler in inverted vertical position must be placed close to the mouth, i.e. at a certain threshold from the middle point of the mouth. This condition verifies that the mouthpiece of the head 320 is at mouth height.

Lastly, unit 171 verifies that the lips are properly closed around the inhaler, i.e. that the distance between the lower middle point and the upper middle point of the mouth (as supplied by unit 160) is less than a certain threshold.

Unit 171 may verify these conditions on successive video frames and only issue a validation of proper positioning when they have been validly verified over a certain number of consecutive video frames.

The stronger or weaker compliance with these thresholds makes it possible to graduate a level, score, indicator or probability that the conditions are verified, that is to say that the inhaler is properly positioned.

Similarly, the use of an automatic learning model makes it possible either to make a binary classification of the video frames as “correct position” or “incorrect position”, or to provide a more nuanced level, score, indicator or probability.

The pressing detection unit 172 verifies whether the actuating finger or fingers are in phase of pressing on the canister 310 of the pressurized metered-dose inhaler. Unit 172 receives as input the 3D marker points of the actuating finger or fingers (from units 162 and 170)

When unit 172 is activated for a phase of pressing detection, it records a reference position of the 3D marker points of the actuating finger or fingers, for example the first position received. This is typically a position without pressing, which, as described below, makes it possible to evaluate the amplitude of the pressing in each later frame.

Unit 172 next determines the movement of the end of the actuating finger or fingers relative to that reference position. For pressing, this is typically determining a relative descending movement of the actuating finger tip relative to a base of the finger (joint of the first phalange to the hand), in comparison with the reference position.

The relative descending movement (longitudinal descending movement, typically vertical) may be compared with a maximum stroke of compression of the inhaler canister.

A maximum real stroke may be obtained through the identification of the pressurized metered-dose inhaler (each inhaler having a known true stroke) may be converted into maximum stroke in the video frame in course of being processed. Thus, the ratio between the measured longitudinal distance of descent of the end of the actuating finger and the frame maximum stroke represents a confidence level, score or indicator or a (so-called pressing) probability that the patient in the video frame is in pressing phase (that is to say pushing in) on the trigger member of the pressurized metered-dose inhaler. This pressing probability, denoted p2 in FIG. 2, is output from unit 172.

This example does not take into account the movement of the end of the actuating finger. More complex models also verifying the movement of the phalanges of the same finger may be taken into account in particular in order to detect (in terms of probability) a particular movement of descending curve of the end of the finger.

As a variant, a set of profiles corresponding to several positions of the fingers according to the intensity of the pressing may be stored in memory and compared to the current frame to determine a profile that is the closest, and hence a pressing amplitude (thus a pressing probability).

As a variant of an algorithm approach, an automatic learning model (trained) may be used.

The compression detection unit 173 gives the compression state of the pressurized metered-dose inhaler. As a matter of fact, the actuation of the inhaler is carried out by mere relative pressing on the canister 310 in the head 320. The analysis of the video frames makes it possible generate a level, score, indicator of confidence or a (so-called compression) probability that the pressurized metered-dose inhaler in a video frame is in a compressed state. This compression probability is denoted p3 in FIG. 2.

Unit 173 receives as input the detection of the inhaler (region of interest identified and inhaler type or family). The inhaler type or family makes it possible to retrieve the real dimension (typically length) of the inhaler in an uncompressed state and its real dimension in a compressed state. This dimensions may be representative of the total length of the inhaler or as a variant of the length of the visible part of the canister. These dimensions are converted into video dimensions in the video frame in course of being processed (for example by multiplying each real length by the ratio between the dimension of the head in the frame and the real dimension of the head 320).

The length measured on the current video frame is then compared with the reference lengths corresponding to the compressed and uncompressed states to attribute (for example in linear manner) a probability comprised between 0 (uncompressed state) and 1 (compressed state).

In a variant, unit 173 implements an automatic learning model, typically a trained neural network, taking as inputs the region of interest around the inhaler and classifying the latter into two categories: inhaler compressed and inhaler uncompressed. Unit 173 may in particular be implemented in conjunction with unit 163, that is to say using the same neural network able to detect an inhaler in a video frame, to categorize that inhaler, to delimit a region of interest around the inhaler and to qualify the state (a probability between 0 and 1 representing the compressed and uncompressed states) of the inhaler for when the inhaler is a pressurized metered-dose inhaler.

In this embodiment, unit 173 takes as input the thumbnail image output from unit 163, containing the inhaler, and yields its probability of being in compressed state. For this, a convolutional neural network for the classification is trained on an image base of compressed and uncompressed inhaler images. The network is chosen with a simple architecture such as LeNet-5 (Y. LeCun, L. Bottou, Y. Bengio, and P. Haffner. Gradient-based learning applied to document recognition. Proceedings of the IEEE, November 1998), and is trained by gradient descent by batches, with a reduction in the learning rate to ensure good convergence of the model.

Unit 174 is a unit for decision as to whether or not synchronization is good between the actuation of the pressurized metered-dose inhaler and an inspiration by the patient. It uses the probabilities of pressing p2, compression p3 and of inhalation p1 corresponding to same instants in time, as described below.

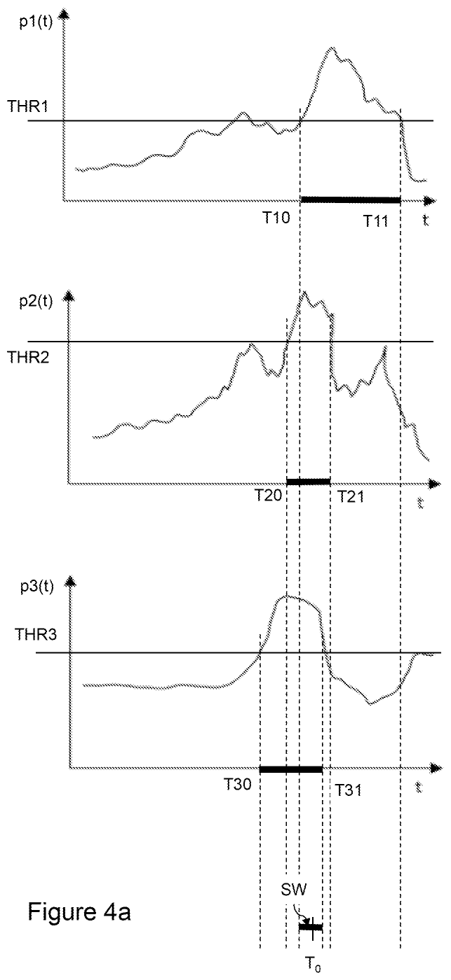

In one embodiment, these probabilities are combined, for example linearly, for each one of a plurality of instants in time. An example of probabilities p1, p2, p3 over time is illustrated in FIG. 4. The sampling of the probability p1 (temporal step between each segment) may be different from that of the probabilities p2 and p3 (frequency of the processed video frames). If required, an interpolation is carried out to obtain a value for the three probabilities at each instant in time considered.

The instants considered may correspond to the smallest sampling period of the three probabilities, thus preferably to each processed frame. Of course, to make the processing lighter, a subset of these instants may be considered.

By way of example, the combined probability at instant t is denoted s(t):

s(t)=a·p1(t)+b·p2(t)+c·p3(t)

It may be optionally averaged over a sliding window of width Tav giving an overall score or a degree of synchronization S(t), as illustrated in FIG. 4. As a variant, averaging over a sliding window may be performed on each of the probabilities p1, p2 and p3 before combination into s(t). In this case, the same window size Tav may be used or as a variant different sizes Tav1, Tav2 and Tav3 of window may be used respectively for the probabilities p1, p2 and p3.

Unit 174 may then compare the overall score with a threshold value THR starting from which a correct synchronization is detected.

The parameters a, b, c, Tav (or Tav1, Tav2 and Tav3) and THR may be learned by cross validation with videos and sound tracks of proper and improper uses.

In the example of FIG. 4, the synchronization score S(t) shows that the patient has carried out proper synchronization between the actuation of the inhaler and his or her inspiration, in the neighborhood of the instant T₀.

If the score S(t) does not exceed the threshold value THR in the analysis window of step 535, it may be determined that the synchronization was not good.

In an embodiment other than the combination of the probabilities into an overall score, it is determined for each probability p1, p2, p3 whether there is a high probability temporal window, respectively for inhalation, pressing and compression. The high probability may simply consist of a threshold value for each probability considered. If several windows are identified for a given probability, the widest may be kept.

With reference to FIG. 4a for example, a threshold THR1 makes it possible to determine a temporal window (T10, T11) in which the inhalation probability is high; a threshold THR2 makes it possible to determine a temporal window (T20, T21) in which the pressing probability is high; and a threshold THR3 makes it possible to determine a temporal window (T30, T31) in which the compression probability is high.

The temporal overlap between the windows is then analyzed to determine a degree of synchronization between the actuation of the inhaler and the patient's inspiration. It is thus a matter of temporally correlating the probabilities previously obtained.

For example, the sub-window SW common to the three temporal windows is determined.

In a variant, the largest sub-window in common between the temporal window (T10, T11) and one of the other two temporal windows is determined. The probability (of inhalation) arising from the audio analysis is thus correlated with a probability arising from the video analysis. This variant makes it possible to overcome possible difficulties in analyzing the compression of the inhaler (for example if it is greatly concealed by the patient's hands) or the pressing by the patient.

The presence of an overlap sub-window for example makes it possible to indicate good synchronization.

In one embodiment, unit 174 verifies that the sub-window has a minimum duration (in particular between 1 s and 3 s) before indicating good synchronization. This reduces the risk of inadvertent detection.

In the example of FIG. 4a, the overlap between the temporal windows shows that the patient has properly synchronized the actuation of the inhaler and his or her inspiration, in the neighborhood of the instant T₀.

In one embodiment, the probabilities p1, p2, p3 are averaged over a predefined temporal window, prior to determination of the temporal windows (T10, T11), (T20, T21) and (T30, T31).

These approaches correlating the probabilities p1, p2, p3 are advantageously robust to the lack of certain probabilities (improper detection in frames for example). Certain missing probabilities may be interpolated from existing probabilities at sufficiently close instants. Similarly, p2 or p3 may be correlated with p1 without the other.

The user device 100 lastly comprises a feedback unit 175 providing feedback to the patient on the analysis of the medication taking. This feedback in particular comprises a signal for the patient of proper use or misuse of the pressurized metered-dose inhaler as determined by unit 174. Other information may be yielded also, for example such as errors detected (improper positioning, inhaler not open, improper expiration/holding of breath, etc.).

Each provision of feedback may be made in real-time or practically in real-time, that is to say when it is generated by a functional unit active during a particular phase of the method described below. As a variant, the provisions of feedback may be provided at the end of the method, in which case they are stored in memory progressively as they are created (during the various phases of the method). The two alternatives may be combined: presentation of the feedbacks upon generation and at the end of the method.

Each provision of feedback may be given visually (screen of the device 100) or orally (loud-speaker) or both.

A indicated above, certain units may be implemented using supervised automatic learning models, typically trained neural networks. The learning of such models from learning data is well-known to the person skilled in the art and is not therefore detailed here. The probabilities generated by the processing units are preferably comprised between 0 and 1, in order to simplify their manipulation, combination and comparison.

Using a flowchart, FIG. 5 illustrates general steps of a method of tracking use or utilization by a patient of a pressurized metered-dose inhaler. These steps use the processing units described above.

This method may for example be implemented by means of a computer program 1030 (application) run by the device 100. By way of example, the patient uses a digital tablet and launches the application according to the invention. This application may propose a step-by-step procedure for guidance (with display of each of the actions to perform as described below) or leave the patient to perform medication taking, without instruction.

The method commences with the launch of the execution of the program. The method enables the program to successively pass into several execution states, each state corresponding to a step. Each state may only be activated if the preceding state is validated (either by positive detection or by expiry of a predefined time or time out). In each state, certain units are active (for the needs of the corresponding step), others not, thereby limiting the use of processing resources.

An indication of the current state may be supplied to the patient, for example the state (that is to say the phase or operation in course of the method) is displayed on the screen. Similarly, feedbacks as to the proper performance of a given phase or as to the existence of an error may be supplied to the patient in real-time, for example displayed on the screen.

At step 500, the video and audio recordings by units 150 and 151 via the camera 105 and the microphone 107 are commenced. Each frame acquired is stored in memory, and the same applies for the audio signal possibly converted into several audio segments.

At step 505, the method enters into the “face detection” state. Unit 160 is activated making it possible to detect a face in the video frames. As soon as a face is detected over several successive video frames (for example a predefined number), the step is validated. Otherwise, the step lasts until expiry of a time out.

The method proceeds to the “inhaler detection” state at step 510. Unit 163 is activated making it possible to detect an inhaler, to locate it and to determine its type or family. This makes it possible to recover useful information for the following steps (maximum stroke, classes of holding the inhaler, etc.).

If the inhaler is not of pressurized metered-dose inhaler type, the method may continue as in the known techniques.

If the inhaler is of pressurized metered-dose inhaler type, its model or its family is recognized and stored in memory.

The method proceeds to the “detection of the remaining doses” state at step 515 if the inhaler model recognized has a dose counter, otherwise (model not recognized or no counter) it proceeds directly to step 520.

At step 515, unit 163 which is still activated carries out tracking of the inhaler over successive video frames, determines a sub-zone of the inhaler corresponding to the indication of the remaining doses (counter or dosimeter). Once this sub-zone has been located, analysis by OCR (optical character recognition) is carried out in order to determine whether a sufficient number of doses remains (for example the value indicated must be different from 0).

In the negative, the method may stop with an error message or continue by storing that error for display at the time of final reporting.

In the affirmative, the method proceeds to the “opening detection” state at step 520. This step implements unit 164 which is activated for that occasion. Again an indicator may be displayed to the patient for as long as unit 164 does not detect that the inhaler is open.

When the opening is detected or after a time out, the method proceeds to the “deep expiration detection” state at step 525. Unit 164 is deactivated. This step 525 implements unit 165 which is activated for that occasion. Unit 165 for example performs temporal correlation between the sound detection of a deep expiration in the audio signal and the detection of an open mouth in the video signal (by unit 160).

The probability (or the confidence score) of expiration is stored in memory to be indicated to the patient in final reporting, in particular on a scale of 1 to 10.

When an expiration has been detected or after a time out (for example the expiration phase is contained within 5 s approximately), the method proceeds to the “detection for proper positioning of the inhaler” state at step 530. Unit 165 is deactivated. This step 530 implements unit 171 described above which is activated for that occasion. It requires the activation of units 161, 162 and 170, unit 160 still being activated. Thus, these first units only begin processing the video frames as of this step.

An indicator may be displayed to the patient indicated to him or her that the inhaler is wrongly positioned, in particular in the wrong orientation or wrongly positioned relative to the patient's mouth.

This indicator may disappear when proper positioning is detected over a number of consecutive video frames. The method then proceeds to the “inhalation synchronization detection” state at step 535.

The method may also pass into this state after expiry of a time out even if proper positioning has not been correctly validated (which will for example be indicated to the patient at the final step 550).

The steps up to this point thus make it possible to determine the right time at which to perform the detection of a proper or improper synchronization of the actuation of the inhaler and of the patient's inspiration/inhalation. This detection step 535 is thus triggered by the detection of proper positioning of the pressurized metered-dose inhaler relative to the patient in the earlier video frames.

The phase of inhalation by the patient lasts in general less than 5 s, for example 3s, thus a time out (of 5 s) for the step may be set up.

The “inhalation synchronization detection” state activates units 167, 172 and 173 for processing the video frames and the audio segments that arrive from this point on, as well as unit 174.

Unit 167 provides the inhalation probabilities p1(t) so long as the step continues. Unit 172 provides the pressing probabilities p2(t). Unit 173 provides the compression probabilities p3(t).

Unit 174 processes, in real-time or after the time out of the step, all the probabilities p1(t), p2(t) and p3(t) in order to determine the degree of synchronization between the actuation one of the pressurized metered-dose inhaler and an inspiration by the patient as described above. This information is stored in memory and/or displayed to the patient, via the feedback unit 175.

In one embodiment, step 535 can include a continuous verification of proper positioning as carried out at step 530. This makes it possible to alert the patient or to store an error in case the patient modifies, in detrimental manner, the positioning of his or her inhaler.

At the end of the time out or in case of detection of a satisfactory degree of synchronization, the method proceeds to the following state of “breath holding detection” at step 540. This is the end of the operation of detecting proper or improper synchronization.

Units 161, 162, 167, 170, 171, 172, 173 may be deactivated, unit 160 being kept active to track the state of opening of the mouth, as well as unit 163. Unit 166 is then activated, processing of the incoming audio segments and/or the new video frames, to determine whether or not the patient is holding his or her breath for a sufficient duration. Step 540 lasts a few seconds (for example 5s) after which units 160 and 166 are deactivated.

The method then proceeds to the “inhaler closing detection” state at step 545. This step uses unit 164 which is again activated to detect the closing of the inhaler.

Time out is provided, in particular because the patient may remove the inhaler from the field of the camera, preventing any detection of closing.

If closing is detected or the time out expires, the method proceeds to the following step 550 in the “reporting” state.

In one embodiment, steps 540 and 545 are carried out in parallel. As a matter of fact, it may be that the patient closes the inhaler at the same time as he or she holds their breath. Units 160, 163, 164 and 166 are then active at the same time.

At step 550, the units that are still active, 163, 164, are deactivated. The feedback unit 175 is activated for needed, which retrieves from memory all the messages/errors/indications stored in memory by the various units activated during the method.

The messages, including that specifying the degree of synchronization between the actuation of the pressurized metered-dose inhaler and an inspiration by the patient, are provided to the patient, for example simply through display on the screen of the program being executed. The reporting may in particular detail the result of each step, with an associated level of success.

Although the above description of the method of FIG. 5 activates and deactivates the units upon request according to the progress of the method, it may be provided that all or some of the units are activated at launch of the program. Typically, the feedback unit 175 may be activated from the outset in order to enable provision of feedback to the patient at any phase of the method. Moreover, units 160, 161, 162 and 163 may also be activated from the outset. Optionally, unit 170 is too. On a subsidiary basis, units 164, 165, 166 are too.

The preceding examples are only embodiments of the invention which is not limited thereto.

1. Computer-implemented method for tracking use, by a patient, of a pressurized metered-dose inhaler, comprising the following steps:
obtaining a video signal and an audio signal of a patient using a pressurized metered-dose inhaler,
calculating, for each of a plurality of video frames of the video signal, at least one from among a so-called pressing probability, that an actuating finger of the patient in the video frame is in a phase of pressing on a trigger member of the pressurized metered-dose inhaler, and a so-called compression probability, that the pressurized metered-dose inhaler in the video frame is in a compressed state,
calculating, for each of a plurality of audio segments of the audio signal, a so-called inhalation probability, of the patient performing, in the audio segment, an inspiration combined with the aerosol stream,
determining a degree of synchronization between the actuation of the pressurized metered-dose inhaler and an inspiration by the patient from the pressing, compression and inhalation probabilities corresponding to same instants in time, and
accordingly issuing to the patient a signal of proper use or misuse of the pressurized metered-dose inhaler.

2. The method according to claim 1, wherein determining a degree of synchronization comprises determining, for each type of probability, a temporal window of high probability, and
the degree of synchronization is a function of a temporal overlap between the temporal windows so determined for the probabilities.

3. The method according to claim 1, wherein determining a degree for synchronization comprises:
combining, for each of a plurality of instants in time, the probabilities of pressing, of compression and of inhalation corresponding to said instant in time into a combined probability, and
determining, from the combined probabilities, a degree of synchronization between the actuation of the pressurized metered-dose inhaler and an inspiration by the patient.

4. The method according to claim 3, wherein determining a degree of synchronization further comprises comparing the combined probabilities with a threshold value of proper synchronization.

5. The method according to claim 1, wherein calculating a pressing probability for a video frame comprises:
detecting, in the video frame, points representing the actuating finger, and
determining a relative descending movement of the tip of the actuating finger relative to a base of the finger, compared to at least one temporally preceding video frame,
the pressing probability being a function of the amplitude of the descending movement from a starting position determined in a preceding video frame.

6. The method according to claim 5, wherein calculating a pressing probability for a video frame comprises comparing the amplitude of the movement to a dimension of the pressurized metered-dose inhaler in the video frame.

7. The method according to claim 1, wherein calculating a compression probability for a video frame comprises:
comparing a length of the pressurized metered-dose inhaler in the video frame with a reference length of the pressurized metered-dose inhaler.

8. The method according to claim 1, wherein calculating an inhalation probability for an audio segment comprises:
converting the audio segment into a spectrogram, and
using the spectrogram as input to a trained neural network which outputs the inhalation probability.

9. The method according to claim 1, wherein the steps consisting of calculating the pressing, compression and inhalation probabilities on later audio segments and video frames are triggered by the detection of proper positioning of the pressurized metered-dose inhaler relative to the patient in earlier video frames.

10. The method according to claim 1, further comprising an initial determination step for determining opening of the metered-dose inhaler by detecting a characteristic click sound in at least one audio segment of the audio signal, the detection employing a learnt detection model.

11. Computer system comprising one or more processors configured for:
obtaining a video signal and an audio signal of a patient using a pressurized metered-dose inhaler,
calculating, for each of a plurality of video frames of the video signal, at least one from among a so-called pressing probability, that an actuating finger of the patient in the video frame is in a phase of pressing on a trigger member of the pressurized metered-dose inhaler, and a so-called compression probability, that the pressurized metered-dose inhaler in the video frame is in a compressed state,
calculating, for each of a plurality of audio segments, a so-called inhalation probability, of the patient performing, in the audio segment, an inspiration combined with the aerosol stream,
determining a degree of synchronization between the actuation of the pressurized metered-dose inhaler and an inspiration by the patient from the pressing, compression and inhalation probabilities corresponding to same instants in time, and
accordingly issuing to the patient a signal of proper use or misuse of the pressurized metered-dose inhaler.

12. Computer-readable non-transient medium storing a program which, when executed by a microprocessor or a computer system, causes the system to carry out the method of claim 1.

13. The method according to claim 2, wherein calculating a pressing probability for a video frame comprises:
detecting, in the video frame, points representing the actuating finger, and
determining a relative descending movement of the tip of the actuating finger relative to a base of the finger, compared to at least one temporally preceding video frame,
the pressing probability being a function of the amplitude of the descending movement from a starting position determined in a preceding video frame.

14. The method according to claim 3, wherein calculating a pressing probability for a video frame comprises:
detecting, in the video frame, points representing the actuating finger, and
determining a relative descending movement of the tip of the actuating finger relative to a base of the finger, compared to at least one temporally preceding video frame,
the pressing probability being a function of the amplitude of the descending movement from a starting position determined in a preceding video frame.

15. The method according to claim 4, wherein calculating a pressing probability for a video frame comprises:
detecting, in the video frame, points representing the actuating finger, and
determining a relative descending movement of the tip of the actuating finger relative to a base of the finger, compared to at least one temporally preceding video frame,
the pressing probability being a function of the amplitude of the descending movement from a starting position determined in a preceding video frame.

16. The method according to claim 2, wherein calculating a compression probability for a video frame comprises:
comparing a length of the pressurized metered-dose inhaler in the video frame with a reference length of the pressurized metered-dose inhaler.

17. The method according to claim 3, wherein calculating a compression probability for a video frame comprises:
comparing a length of the pressurized metered-dose inhaler in the video frame with a reference length of the pressurized metered-dose inhaler.

18. The method according to claim 4, wherein calculating a compression probability for a video frame comprises:
comparing a length of the pressurized metered-dose inhaler in the video frame with a reference length of the pressurized metered-dose inhaler.

19. The method according to claim 5, wherein calculating a compression probability for a video frame comprises:
comparing a length of the pressurized metered-dose inhaler in the video frame with a reference length of the pressurized metered-dose inhaler.

20. The method according to claim 6, wherein calculating a compression probability for a video frame comprises:
comparing a length of the pressurized metered-dose inhaler in the video frame with a reference length of the pressurized metered-dose inhaler.
